(19) United States
(12) Patent Application Publication
(10) Pub. No.: US 2025/0364142 A1
(43) Pub. Date: Nov. 27, 2025

(54) NEONATAL INTENSIVE CARE UNIT-SPECIFIC BIG DATA-BASED NEONATAL ACUTE KIDNEY INJURY PREDICTION ARTIFICIAL INTELLIGENCE SYSTEM

(71) Applicants: JEONBUK NATIONAL UNIVERSITY HOSPITAL, Jeonbuk-do (KR); INDUSTRIAL COOPERATION FOUNDATION JEONBUK NATIONAL UNIVERSITY, Jeonbuk-do (KR)

(72) Inventors: HYUN HO KIM, Jeonbuk-do (KR); HYUNG MIN JUN, Jeonbuk-do (KR); MYOUNG-HWAN KO, Jeonbuk-do (KR)

(21) Appl. No.: 18/883,008

(22) Filed: Sep. 12, 2024

(30) Foreign Application Priority Data
May 23, 2024 (KR) 10-2024-0066966

(51) Int. Cl. G16H 50/30; A61B 5/00; A61B 5/0205; A61B 5/20; G16H 10/60; G16H 50/20

(52) U.S. Cl. CPC G16H 50/30; A61B 5/0205; A61B 5/201; A61B 5/7264; A61B 5/7275; G16H 10/60; G16H 50/20; A61B 2503/045

(57) ABSTRACT

A neonatal intensive care unit-specific big data-based neonatal acute kidney injury prediction artificial intelligence system enables rapid and clear diagnosis of neonatal acute kidney injury, which has high morbidity and mortality rates. The neonatal intensive care unit-specific big data-based neonatal acute kidney injury prediction artificial intelligence system guides doctors to make clinical decision by performing deep learning calculations on an EMR (electronic medical record) data-based disease prediction model and a biosignal-based disease prediction model that are output by acquiring EMR data and clinical observation data from a neonatal intensive care unit.

### CROSS-REFERENCE TO RELATED APPLICATION AND CLAIM OF PRIORITY

This application claims the benefit under 35 USC § 119 of Korean Patent Application No. 10-2024-0066966, filed on May 23, 2024, in the Korean Intellectual Property Office, the entire disclosure of which is incorporated herein by reference for all purposes

### BACKGROUND

### 1. Technical Field

The present disclosure relates to a neonatal intensive care unit-specific big data-based neonatal acute kidney injury prediction artificial intelligence system, and is directed to enabling rapid and clear diagnosis of neonatal acute kidney injury disease, which has high disease morbidity and mortality rates, by preparing and providing a neonatal intensive care unit-specific big data-based neonatal acute kidney injury prediction artificial intelligence system that guides doctors to make clinical decision by performing deep learning calculations on an EMR (electronic medical record) data-based disease prediction model and a biosignal-based disease prediction model that are output by acquiring medical data and clinical observation data from a neonatal intensive care unit.

### 2. Background Art

Generally, a neonatal intensive care unit is a specialized medical facility to manage serious medical conditions in newborns or complications resulting from premature birth.

The medical environment of the neonatal intensive care unit is experiencing a serious manpower shortage due to the recent rapid decline in the number of pediatric and adolescent specialists. Also, in addition to the rapid decline in the number of specialists, there is a severe shortage of neonatal subspecialists.

In this environment where there is a shortage of pediatric specialists, acute kidney injury occurs in 30% of premature babies, 48% at 22-28 weeks, 18% at 29-35 weeks, and 37% at 36-37 weeks. The cause is the vulnerability of premature babies' kidneys, and there is a need for intensive medical care in the intensive care unit.

Due to this poor medical environment, newborns hospitalized in the neonatal intensive care unit, especially very low birth weight infants, are vulnerable to various complications due to their complex physiological characteristics and immature organ functions. When acute kidney injury occurs, the mortality rate increases by 2.5-8.3 times, and the length of hospitalization increases by 8.8 days.

Meanwhile, the diagnostic criteria for acute kidney injury in newborns are mainly based on an increase in serum creatine level or a decrease in urine volume, but diagnosis is not easy due to difficulties in blood collection and urine volume measurement.

### SUMMARY

Accordingly, the present disclosure is intended to solve the problem of not being able to quickly and clearly diagnose neonatal acute kidney injury, which has high morbidity and mortality rates.

That is, the present disclosure includes a neonatal intensive care unit-specific big data-based neonatal acute kidney injury prediction artificial intelligence system that guides doctors to make clinical decision by performing deep learning calculations on an EMR-based disease prediction model and a biosignal-based disease prediction model that are output by acquiring EMR data and clinical observation data from a neonatal intensive care unit.

To this end, the neonatal intensive care unit-specific big data-based neonatal acute kidney injury prediction artificial intelligence system according to the present disclosure includes an EMR-based disease prediction model unit, a biosignal-based disease prediction model unit and a clinical diagnosis supporting system unit;

the EMR-based disease prediction model unit is configured to output an EMR-based disease prediction model by performing deep learning on medical data of normal newborns and medical data of newborns with acute kidney injury and death newborns through medical data of initial and re-examination records, surgical records, nursing records, and discharge records recorded and computerized in the neonatal intensive care unit;

the biosignal-based disease prediction model unit is configured to output a biosignal-based disease prediction model that outputs a disease prediction model using biosignals of electrocardiogram, oxygen saturation, and blood pressure generated and computerized in the neonatal intensive care unit; and the clinical diagnosis supporting system unit is configured to perform deep learning on the EMR-based disease prediction model output from the EMR-based disease prediction model unit and the biosignal-based disease prediction model output from the biosignal-based disease prediction model unit and provide a proposal so that a doctor quickly and clearly diagnoses neonatal acute kidney injury and death early and takes an appropriate treatment measure.

Therefore, the present disclosure enables rapid and clear diagnosis of neonatal acute kidney injury disease, which has high disease morbidity and mortality rates, by preparing and providing a neonatal intensive care unit-specific big data-based neonatal acute kidney injury prediction artificial intelligence system that guides doctors to make clinical decision by performing deep learning calculations on an EMR-based disease prediction model and a biosignal-based disease prediction model that are output by acquiring medical data and clinical observation data from a neonatal intensive care unit.

### BRIEF DESCRIPTION OF THE DRAWINGS

FIGS. 1 and 2 are block diagrams exemplarily showing main components of the present disclosure.

FIGS. 3 to 4 are diagrams exemplarily showing a big data-based neonatal acute kidney injury prediction artificial intelligence system according to the present disclosure.

FIG. 5 is a diagram exemplarily showing an EMR-based disease prediction model unit according to an embodiment of the present disclosure.

FIG. 6 is a diagram exemplarily showing a biosignal-based disease prediction model unit according to an embodiment of the present disclosure.

FIG. 7 is a diagram exemplarily showing a clinical diagnosis supporting system unit according to an embodiment of the present disclosure.

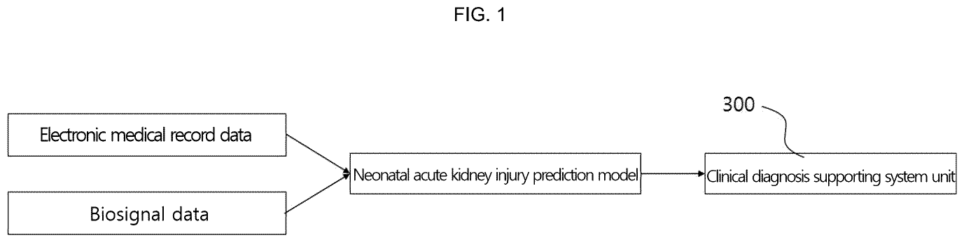

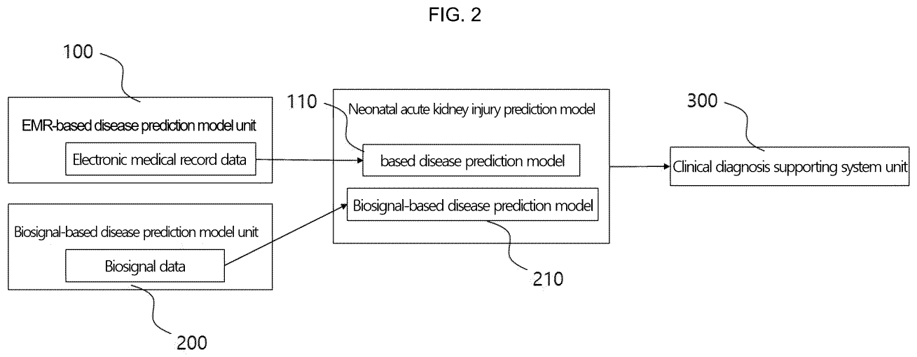

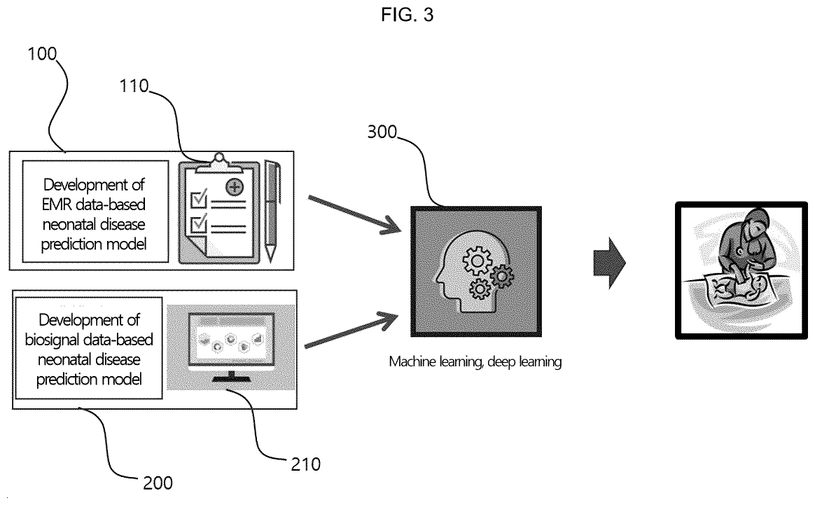

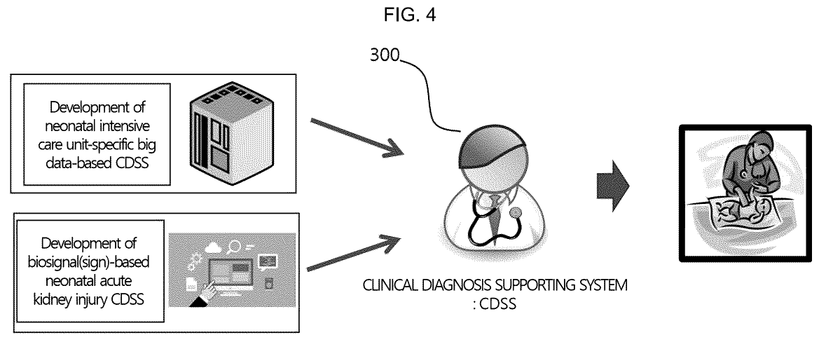

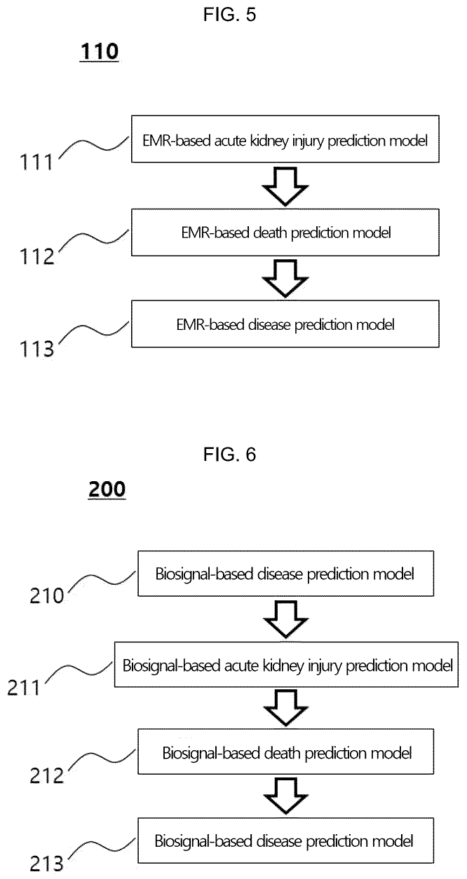

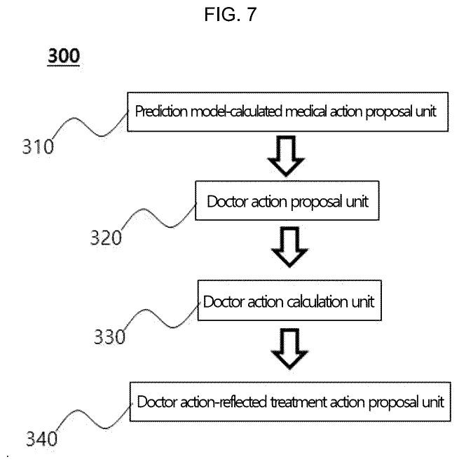

### DETAILED DESCRIPTION

Hereinafter, the present disclosure will be described in detail with reference to the attached drawings.

The present disclosure enables rapid and clear diagnosis of neonatal acute kidney injury disease, which has high morbidity and mortality rates.

Hereinafter, preferred embodiments of the present disclosure will be described in detail with reference to the accompanying drawings. Prior to the description, it should be understood that the terms used in the specification and the appended claims should not be construed as limited to general and dictionary meanings, but interpreted based on the meanings and concepts corresponding to technical aspects of the present disclosure on the basis of the principle that the inventor is allowed to define terms appropriately for the best explanation.

Therefore, the description proposed herein is just a preferable example for the purpose of illustrations only, not intended to limit the scope of the disclosure, so it should be understood that other equivalents and modifications could be made thereto without departing from the scope of the disclosure.

That is, the present disclosure includes a neonatal intensive care unit-specific big data-based neonatal acute kidney injury prediction artificial intelligence system that guides doctors to make clinical decision by performing deep learning calculations on an EMR (electronic medical record) data-based disease prediction model and a biosignal-based disease prediction model that are output by acquiring EMR data and clinical observation data from a neonatal intensive care unit.

The neonatal intensive care unit-specific big data-based neonatal acute kidney injury prediction artificial intelligence system includes an EMR-based disease prediction model unit 100, a biosignal-based disease prediction model unit 200 and a clinical diagnosis supporting system unit 300.

Here, the EMR-based disease prediction model unit 100 outputs an EMR-based disease prediction model 110 by performing deep learning on medical data of normal newborns and medical data of newborns with acute kidney injury and death newborns through medical data of initial and re-examination records, surgical records, nursing records, and discharge records recorded and computerized in the neonatal intensive care unit.

The EMR-based disease prediction model 110 includes an EMR-based acute kidney injury prediction model 111 that outputs a neonatal acute kidney injury prediction model, an EMR-based death prediction model 112 that outputs a neonatal death prediction model, and an EMR-based disease prediction model 113 that outputs a neonatal death or acute kidney injury prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination.

In addition, the biosignal-based disease prediction model unit 200 outputs a biosignal-based disease prediction model 210 that outputs a disease prediction model using biosignals of electrocardiogram, oxygen saturation, and blood pressure generated and computerized in the neonatal intensive care unit.

The biosignal-based disease prediction model 210 includes a biosignal-based acute kidney injury prediction model 211 that outputs a neonatal acute kidney injury prediction model, a biosignal-based death prediction model 212 that outputs a neonatal death prediction model, and a biosignal-based disease prediction model 213 that outputs a neonatal death or acute kidney injury prediction model using the biosignals of electrocardiogram, oxygen saturation, and blood pressure generated and computerized in the neonatal intensive care unit.

In addition, the clinical diagnosis supporting system unit 300 includes suggesting a neonatal acute kidney injury and death decision-making process, and is configured to perform deep learning on the EMR-based disease prediction model 110 output from the EMR-based disease prediction model unit 100 and the biosignal-based disease prediction model 210 output from the biosignal-based disease prediction model unit 200 and provide a proposal so that a doctor quickly and clearly diagnoses neonatal acute kidney injury and death early and takes an appropriate treatment measure. For example, the treatment measure includes, but not limited to supportive care including treating the underlying cause, such as sepsis with antibiotics, or relieving an obstruction, a fluid therapy including correcting hypovolemia, and using vasopressors to maintain blood pressure and tissue perfusion, a fluid balance including using fluid restriction, diuretics, or renal replacement therapy to manage fluid overload, electrolyte supplementation or binders to restore electrolyte and acid-base homeostasis, providing adequate nutrition to promote healing, a kidney replacement therapy including peritoneal dialysis, continuous renal replacement therapy (CRRT), or intermittent hemodialysis (IHD), caffeine exposure in the first postnatal week may be associated with a decreased odds of AKI, etc.

The clinical diagnosis supporting system unit 300 includes a prediction model-calculated medical action proposal unit 310 configured to provide a treatment action proposal to a doctor according to the EMR-based disease prediction model 110 of the EMR-based disease prediction model unit 100 and the biosignal-based disease prediction model 210 of the biosignal-based disease prediction model unit 200; a doctor action proposal unit 320 configured to allow the doctor to input a medical action proposal in response to the treatment action proposal of the prediction model-calculated medical action proposal unit 310; a doctor action calculation unit 330 configured to output a doctor action-reflected treatment action proposal by calculating the medical treatment proposal of the doctor proposed through the doctor action proposal unit 320 to reflect the treatment action proposal proposed in the prediction model-calculated medical action proposal unit 310; and a doctor action-reflected treatment action proposal unit 340 configured to deliver the doctor action-reflected treatment action proposal output through the doctor action calculation unit 330 to the doctor.

Hereinafter, the effects of the present disclosure will be explained.

By applying the present disclosure including a neonatal intensive care unit-specific big data-based neonatal acute kidney injury prediction artificial intelligence system that guides doctors to make clinical decision by performing deep learning calculations on a EMR-based disease prediction model and a biosignal-based disease prediction model that are output by acquiring EMR data and clinical observation data from a neonatal intensive care unit, wherein the neonatal intensive care unit-specific big data-based neonatal acute kidney injury and death prediction artificial intelligence system includes an EMR-based disease prediction model unit 100, a biosignal-based disease prediction model unit 200 and a clinical diagnosis supporting system unit 300, wherein the EMR-based disease prediction model 110 includes an EMR-based acute kidney injury prediction model 111 that outputs a neonatal acute kidney injury prediction model through medical data of a medical record sheet containing hospitalization records, progress records, surgical records, nursing records, and discharge records recorded and computerized in the neonatal intensive care unit and a clinical observation record sheet containing intake, excretion, physical measurements, and examination, an EMR-based death prediction model 112 that outputs a neonatal death prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination, and an EMR-based disease prediction model 113 that outputs a neonatal death or acute kidney injury prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination, wherein the biosignal-based disease prediction model 210 includes a biosignal-based acute kidney injury prediction model 211 that outputs a neonatal acute kidney injury prediction model using the biosignals of electrocardiogram, oxygen saturation, and blood pressure generated and computerized in the neonatal intensive care unit and a biosignal-based death prediction model 212 that outputs a neonatal death prediction model using the biosignals of electrocardiogram, oxygen saturation, and blood pressure generated and computerized in the neonatal intensive care unit, wherein the clinical diagnosis supporting system unit 300 includes a prediction model-calculated medical action proposal unit 310 configured to provide a treatment action proposal to a doctor according to the EMR-based disease prediction model 110 of the EMR-based disease prediction model unit 100 and the biosignal-based disease prediction model 210 of the biosignal-based disease prediction model unit 200, a doctor action proposal unit 320 configured to allow the doctor to input a medical action proposal in response to the treatment action proposal of the prediction model-calculated medical action proposal unit 310, a doctor action calculation unit 330 configured to output a doctor action-reflected treatment action proposal by calculating the medical treatment proposal of the doctor proposed through the doctor action proposal unit 320 to reflect the treatment action proposal proposed in the prediction model-calculated medical action proposal unit 310, and a doctor action-reflected treatment action proposal unit 340 configured to deliver the doctor action-reflected treatment action proposal output through the doctor action calculation unit 330 to the doctor, it is possible to rapidly and clearly diagnose neonatal acute kidney injury disease, which has high disease morbidity and mortality rates.

What is claimed is:

1. A neonatal intensive care unit-specific big data-based neonatal acute kidney injury prediction artificial intelligence system, comprising:
an electronic medical record (EMR)-based disease prediction model unit;
a biosignal-based disease prediction model unit; and
a clinical diagnosis supporting system unit,
wherein the EMR-based disease prediction model unit is configured to output an EMR-based disease prediction model by performing deep learning on medical data of normal newborns and medical data of newborns with acute kidney injury and death newborns through medical data of a medical record sheet containing hospitalization records, progress records, surgical records, nursing records, and discharge records recorded and computerized in the neonatal intensive care unit and a clinical observation record sheet containing intake, excretion, physical measurements, and examination;
wherein the biosignal-based disease prediction model unit is configured to output a biosignal-based disease prediction model that outputs a disease prediction model using biosignals of electrocardiogram, oxygen saturation, and blood pressure generated and computerized in the neonatal intensive care unit;
wherein the clinical diagnosis supporting system unit is configured to perform deep learning on the EMR-based disease prediction model output from the EMR-based disease prediction model unit and the biosignal-based disease prediction model output from the biosignal-based disease prediction model unit and provide a proposal so that a doctor quickly and clearly diagnoses neonatal acute kidney injury and death early and takes an appropriate treatment measure,
wherein the neonatal intensive care unit-specific big data-based neonatal acute kidney injury prediction artificial intelligence system guides a doctor to make clinical decision by performing deep learning calculations on the EMR-based disease prediction model and the biosignal-based disease prediction model that are output by acquiring medical data and clinical observation data from a neonatal intensive care unit.

2. The neonatal intensive care unit-specific big data-based neonatal acute kidney injury prediction artificial intelligence system according to claim 1, wherein the EMR-based disease prediction model includes:
an EMR-based acute kidney injury prediction model that outputs a neonatal acute kidney injury prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination;
an EMR-based death prediction model that outputs a neonatal death prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination; and
an EMR-based disease prediction model that outputs a neonatal death or acute kidney injury prediction model through the medical data of the medical record sheet containing hospitalization records, progress records, surgical records, nursing records, and discharge records recorded and computerized in the neonatal intensive care unit and the clinical observation record sheet containing intake, excretion, physical measurements, and examination.

3. The neonatal intensive care unit-specific big data-based neonatal acute kidney injury prediction artificial intelligence system according to claim 1, wherein the biosignal-based disease prediction model includes:
a biosignal-based acute kidney injury prediction model that outputs a neonatal acute kidney injury prediction model using the biosignals of electrocardiogram, oxygen saturation, and blood pressure generated and computerized in the neonatal intensive care unit;
a biosignal-based death prediction model that outputs a neonatal death prediction model using the biosignals of electrocardiogram, oxygen saturation, and blood pressure generated and computerized in the neonatal intensive care unit; and
a biosignal-based disease prediction model that outputs a neonatal death or acute kidney injury prediction model using the biosignals of electrocardiogram, oxygen saturation, and blood pressure generated and computerized in the neonatal intensive care unit.

4. The neonatal intensive care unit-specific big data-based neonatal acute kidney injury prediction artificial intelligence system according to claim 1, wherein the clinical diagnosis supporting system unit includes:
a prediction model-calculated medical action proposal unit configured to provide a treatment action proposal to a doctor according to the EMR-based disease prediction model of the EMR-based disease prediction model unit and the biosignal-based disease prediction model of the biosignal-based disease prediction model unit;
a doctor action proposal unit configured to allow the doctor to input a medical action proposal in response to the treatment action proposal of the prediction model-calculated medical action proposal unit;
a doctor action calculation unit configured to output a doctor action-reflected treatment action proposal by calculating the medical treatment proposal of the doctor proposed through the doctor action proposal unit to reflect the treatment action proposal proposed in the prediction model-calculated medical action proposal unit; and
a doctor action-reflected treatment action proposal unit configured to deliver the doctor action-reflected treatment action proposal output through the doctor action calculation unit to the doctor.
